(19) United States
(12) Patent Application Publication
(10) Pub. No.: US 2019/0004596 A1
(43) Pub. Date: Jan. 3, 2019

(54) HANDS-FREE INPUT METHOD AND INTRA-ORAL CONTROLLER APPARATUS

(71) Applicant: SAMSUNG ELETRÔNICA DA AMAZÔNIA LTDA., Campinas (BR)

(72) Inventors: ANTÔNIO HENRIQUE BARBOSA POSTAL, CAMPINAS (BR); JOSÉ MARIA EWERTON DOS SANTOS JÚNIOR, CAMPINAS (BR)

(73) Assignee: SAMSUNG ELETRÔNICA DA AMAZÔNIA LTDA., Campinas (BR)

(21) Appl. No.: 15/715,704

(22) Filed: Sep. 26, 2017

(30) Foreign Application Priority Data
Jun. 29, 2017 (BR) 1020170141969

(51) Int. Cl. G06F 3/01; A61B 5/00; G06F 3/0487; H04W 4/00; G02B 27/01

(52) U.S. Cl. CPC G06F 3/011; A61B 5/682; G02B 27/0176; H04W 4/008; G06F 3/0487

(57) ABSTRACT

A wireless and wearable hands-free device and user interface (UI) method for controlling a pointer interface in the screen or controlled device based on jaw and/or tongue movements and actions may be used by persons with different grades of physical disabilities, on upper members, from lighter to more severe cases, and may also be used for non-handicapped regular users as a new hands-free, almost-invisible interface method.

### CROSS-REFERENCE TO RELATED APPLICATIONS

This application claims the foreign priority benefit of Brazilian Patent Application No. 10 2017 014196 9, filed on Jun. 29, 2017 in the Brazilian Intellectual Property Office, the disclosure of which is incorporated herein by reference.

### BACKGROUND

### 1. Field

The following description refers to a new wireless and wearable hands-free device and user interface (UI) method for controlling a pointer interface in the screen or equipment based on jaw and/or tongue movements and actions. It applies to persons with different grades of physical disabilities, on upper members, from lighter to more severe cases, and it can also be used for non-handicapped regular users as a new hands-free, almost-invisible interface method.

### 2. Description of Related Art

Many user interfaces (UIs) have been proposed for users with physical disabilities on the upper members, in order to help them to control equipment, either directly or through a pointer on a display in a manner similar to that used on a conventional computer mouse.

These UI solutions in prior-art include: eye tracking, use of tongue, use of lips, breath actuators, vertical movement of lower jaw, brain sensing.

Some of these present practical limitations: inconvenience due to the difficulties of actuating or inaccuracy due to difficulties to control an UI with reasonable accuracy. Other aesthetical, functional limitations verified are: the need of external apparatus, frequently around the head or neck which also prevent the use of other medical equipment and the contact between user's mouth parts and external parts of device which may cause hygienic issues.

In common cases of physical disability on the upper members, users do not have full use of their hands, while in the more severe cases the user can only control body parts above the neck. Solutions which requires upper body, neck and head movements, such as moving head rotation, moving head or upper body are not suitable for the last case.

Many solutions propose user interfaces for users with physical disabilities on the uppers members in order help them to control equipment or devices, either directly or through a pointer or cursor on display in a manner similar to that of a conventional computer mouse. These solutions include: eye tracking, use of tongue, use of lips, breath actuator, vertical movement of lower jaw detected by external apparatus, brain sensing.

The most severe cases include amputees, quadriplegics, users with damages on spinal cord by birth or due to an accident, severe cases of arthritis and muscular dystrophy, a degenerative condition which progressively reduces user's motion control and in which face and mouth are the latest parts affected.

The following prior-art documents are closer to the present disclosure but they do not gather the advantages listed below.

The patent document US 2012/0259554 A1 titled “Tongue Tracking Interface Apparatus and Method for Controlling a Computer Program”, by Sony Comp Entertainment Inc., filed on Apr. 8, 2011, describes a tongue tracking interface apparatus for control of a computer program including a mouthpiece configured to be worn over one or more teeth. The mouthpiece can include one or more sensors configured to determine one or more tongue orientation characteristics of the user. The present disclosure also describes an intra-oral device and include user's tongue sensors, but it differs from document US 2012/0259554 A1, since it does not require the user's tongue to be oriented, the tongue rests on longitudinal orientation, just requiring the touch of any of its side parts or tip on the contacts installed on the internal side of the intra-oral arch-shaped device covering user's teeth and placed on the user's dental arches. The patent document US 2012/0259554 A1 describes that the sensors are “located on the front side of the mouthpiece configured so that the sensors sit in front of the user's teeth when the mouthpiece is worn” [0020], while the present disclosure describes sensors that are located in the internal sides of intra-oral device and, consequently, facing the back side of user's teeth and easier for the user's tongue to reach them. Moreover, the present disclosure describes an intra-oral device which contains contacts installed on upper and lower arches, which are easier and more intuitive to differentiate between top and bottom tongue movements; and finally, the present disclosure allows more flexibility of configuration and use by combining tongue and lower jaw movements while using the same intra-oral device and allows the configurable mapping between user's movements and controlled device's movements and actions.

The patent document WO 03/013402 A1 titled “A Tongue and Jaw Operable Control Apparatus”, by Divkey Pty Ltd, filed on Aug. 8, 2001, describes a tongue and jaw operable control apparatus which includes an external body or housing and support means in the form of a multifunctional mouth piece, also placed externally to the mouth. The present disclosure is also based on jaw and tongue movements but it differs from the cited patent because it describes a device completely internal to the mouth which is more convenient to use, more discrete and more hygienic, not requiring mouth contact with external objects. In addition, the intra-oral device of the present disclosure allows the simultaneous use with external equipment, for example the use of respiratory mask in medical devices such as BiPAP (Bilevel Positive Airway Pressure), needed for muscular dystrophy and similar conditions.

The patent document U.S. Pat. No. 8,047,964 B2 titled “Methods and Systems for Lingual Movement to Manipulate an Object”, by Youhanna Al-Tawil, filed on Apr. 22, 2011, describes an intra-oral mouthpiece which has a plurality of pressure sensors for sensing lingual movements in order to manipulate mechanical objects or through a cursor on a display. The present disclosure also includes user's tongue movements but it differs from the cited patent in the following aspects: the patent document U.S. Pat. No. 8,047,964 describes a mouthpiece which is placed over the tongue surface, which occupies space on mouth cavity and prevents user to speak while using it This disclosure's mouthpiece is less invasive and more comfortable, where sensors attached to a thin plate are positioned on the s\ide of the teeth, not occupying significant space inside the mouth cavity and allowing user to speak while using it. Moreover, the present disclosure allows more flexibility of configuration and use by combining tongue and lower jaw movements while using the same intra-oral device and allows the configurable mapping between user's movements and controlled device's movements and actions; and the intra-oral device of the present disclosure allows the simultaneous use with external medical equipment, for example the use of respiratory devices, needed for muscular dystrophy and similar conditions.

The patent document U.S. Pat. No. 4,486,630 A titled “Device for Use by Quadriplegics to Operate a Computer, Video Game or the Like by the Use of Movements of the Jaw and Eyebrows”, by John E. Fetchko, filed on Mar. 11, 1983, describes a headset worn around the head to detect movements of the jaw and/or eyebrows through electric contacts, where the jaw movements are detected by a support attached to user's chin. The present disclosure is also based on jaw movements, besides tongue movements, but it differs from the cited patent by presenting an intra-oral, wearable, wireless device rather than an external device to detect the lower jaw movements, and it includes sensors positioned on the side of an intra-oral device, being more convenient, almost-invisible, lighter and more convenient to use than the cited patent. The present disclosure also combines jaw and tongue movements in unique interface.

The patent document CA 2718303 titled “Inter-oral Touchpad”, by Mathew William Osterloo, filed on Oct. 12, 2010, describes an intra-oral touchpad placed in the mouth with the touch sensitive pad sequestered vertically between the outer teeth and the inner cheek where the tongue is able to contact the exposed touch sensitive surface of the touchpad. The present disclosure is also based on tongue movements but it differs from document CA 2718303 by detecting tongue movements using sensors positioned on the internal side of the intra-oral device composed of thin plaques covering user's teeth and attached to the user's dental arches, rather than a touchable surface, with the advantage of being more discrete and more convenient by not requiring the user to keep jaws separated in order to reach the touch surface close the inner cheek. Also, the present disclosure combines simultaneous tongue and/or lower jaw movements and allows them to be composed and mapped to movements to be performed by a controlled device which can be configured through a movements map setting UI.

The paper and related project titled: “Tongue Drive: A Wireless Brain-Tongue-Computer Interface” by Georgia Tech Research Corporation utilizes a small magnet attached to the tongue and magnetic sensors mounted to headgear, tongue movements are transmitted to a portable computing device and translated into commands. There are two derived projects “A Wireless Magnetoresistive Sensing System for an Intraoral Tongue-Computer Interface”. that reads the magnetic field variations inside the mouth from four 3-axial magneto resistive sensors and “An Arch-Shaped Intraoral Tongue Drive System with Built-in Tongue-Computer Interfacing SoC” which presents an arch-shaped intraoral device which detects magnetic sensor data and recognizes the intended user commands by the sensor signal processing (SSP) algorithm running in a smartphone, and delivers the classified commands to the target devices, such as a personal computer or a powered wheelchair. Differently, the present disclosure does not require a magnet to be installed on user's tongue and do not rely only on magnet sensors, but it proposes punctual contacts placed on the internal sides of arches, touchable by the tongue, and it also extends to detection of jaws movements besides tongue using the same device and it relies on different types of sensors: mechanical, electro-magnetic and optical.

The patent document TWM488060 titled “Computer Input Device for Muscular Dystrophy Patient”, by Univ China SCI & Tech, filed on Jun. 13, 2014, describes an input device based on head movement and eye-tracking. The present disclosure also addresses muscular dystrophy, besides other severe disabilities, but it differs from document TWM488060 by using a different approach and detecting user's jaw and tongue movements through sensors positioned on internal part of the intra-oral device composed of thin plaques covering user's teeth and attached to the user's dental arches, with the advantage of being more discrete than using head movements, being able to help users which cannot move head and providing a more accurate control than eye tracking, prone to involuntary movements.

The patent document JP 4501134 titled “Input Device for Computer”, by Okabe Hiromichi, filed on Nov. 10, 2014, describes an external device with a track ball positioned at the front lower part of the face of an operator, wherein the rotation of the ball is operated by the lower lip of the operator. The present disclosure also addresses users with motor disabilities, but it differs from document JP 4501134 by detecting user's jaw and tongue movements by sensors positioned on internal part of the intra-oral device composed of thin plaques covering user's teeth and attached to the user's dental arches, which is a device completely internal to the mouth, more convenient to use, more discrete and more hygienic, by not requiring mouth contact with external objects. In addition, the intra-oral device of the present disclosure allows the simultaneous use with external medical equipment, for example the use of respiratory devices, needed for muscular dystrophy and similar conditions.

The paper titled “Human-Computer Interface Controlled by the Lip”, by University of São Paulo, filed on January, 2015, proposes human-computer interface controlled by the lower lip, for users with tetraplegia, by using an external head-mounted device which includes a joystick support for the lower lip touch. The present disclosure differs from said paper by detecting user's lower jaw and tongue movements by using sensors positioned on internal part of the intra-oral device which is composed of thin plaques covering user's teeth and attached to the user's dental arches, with the advantage of being more discrete and it is possible to be used with respiratory equipment. In addition, the present disclosure does not present hygienic issues, by not requiring mouth contact with an external device.

The method and apparatus of the present disclosure disclose a new solution to overcome the cited limitations and can be applied for different grades of physical disabilities, which is based on an intraoral device similar to dental plaques in shape of arches, denture or braces used on Dentistry as films attached to tooth surface on the upper and lower dental arches, which can detect user inputs from the lower jaw and tongue movements through sensors installed on the plaques, and wirelessly communicate with a smartphone, PC, notebook, smart TV, head-mounted display (HMD) for virtual reality/augmented reality (VR/AR) or other devices and to control a UI or a pointer device on the display. These controlled devices motions can be configured to user's lower jaw and tongue movements according to its type and interpretation.

For the most severe conditions where users have a high dependency of constant assistance, the subject matter of the present disclosure will significantly reduce the degree of dependency of the user from a care-taker or family member. These users enjoy online interacting with friends and family and adequate input methods are welcome. Even in the critical cases, oral parts movements are preserved or, in case of degenerative conditions, these parts are the latest ones to be affected.

The present disclosure relates to a hands-free input method for controlling electronic devices based on user's jaw and tongue movements comprising the following steps:

a) user uses a movement map configuration UI to map lower jaw movements and selection of the corresponding movements of a controlled device;

b) connecting an intra-oral device to the controlled device using a wireless communication method available;

c) detecting the jaws' movement by means of jaw sensors and the tongue movement by means of tongue sensors;

d) the micro-controlled system look-ups the sensed user's jaw movement in the configuration UI;

e) transmitting the corresponding movement to the controlled device; and

f) the controlled device performs the movement in the corresponding direction or perform the selected action, e.g. the “Selection” action.

Further, the present disclosure is also defined by an intra-oral apparatus for controlling mobile devices based on user's jaw and tongue movements, the apparatus comprising:
an upper and lower device arches attachable to the user's dental arches, covering user's teeth;
sensors installed on the device arches responsible for detecting the user's lower jaw and tongue movements;
a wireless data communicating system embedded in the device arches which can be connected to the controlled device;
a configuration user interface (UI) responsible for mapping user jaw and tongue movements and actions to corresponding movements and actions on the controlled device, either directly or through a pointer on the controlled device's display; and
a micro-controlled system embedded in the device arches executing programmable instructions, reading sensors measurements, looking-up the predefined movements map for the user's lower jaw and tongue movement or action, and wirelessly transmitting the corresponding mapped movement or action to the be executed by the controlled device.

The method and apparatus of the present disclosure is also extended to people without disabilities as a new UI, for hands-free use. This is a strong opportunity and trend: the use of “invisibles” (invisible devices) beyond the “wearables”, also related to “Body Area Networks” (BAN) where “BAN devices may be embedded inside the body, implants, may be surface-mounted on the body in a fixed position”.

The method and apparatus of the present disclosure apply as a controller for devices and equipment with or without display, such as powered wheelchairs, toys, car, kart or robots.

In a specific embodiment, the concept applies to Immersive Reality technologies, more specifically AR/VR HMD's (Head-mounted display) devices, including VR devices embedding a smartphone (e.g. Samsung Gear VR, Google Cardboard) and AR pass-through glasses (e.g. MS HoloLens, Google Glass).

In this embodiment, the controlled equipment is the HMD device wherein the user's jaw and tongue movements are reflected to movements of virtual objects or pointer to virtual objects in the Virtual Environment.

When using such HMD devices, user cannot easily reach a touchscreen and he has few input methods available; for these cases hands-free methods are very convenient for the user to interact with the virtual content while having hands available for executing other activities. According to one embodiment, user can control virtual objects in AR/VR environments by using jaw and tongue movements combined.

Specifically, the present disclosure is related to the accessibility, wearables, user experience (UX) and augmented reality (AR)/virtual reality (VR) areas. It presents a wearable, wireless, arch-shaped, intra-oral device and hands-free input method which targets the following users and scenarios:
physically handicapped users with different grades of disabilities, from lighter to more severe cases;
non-handicapped (“regular”) users; and
users interacting with virtual environments (AR/VR).

The present disclosure relates to a new controller apparatus which is operable by user's lower jaw and/or tongue and a hands-free input method to be used by people with different grades of disabilities to control equipment, either directly or through a pointer or a cursor on the display of a device such as a smartphone, a PC, a notebook, a smart TV or a VR/AR head-mounted display (HMD). Besides those devices, controlled equipment also includes powered wheelchairs, cars and robots, not limited to these.

The apparatus is based on an intraoral device similar to dental plaques in format of arches, similar to denture or braces used on Dentistry as films comfortably attached to tooth surfaces, on the upper and lower dental arches, which can detect user inputs from lower jaw and tongue movements through sensors installed on the intraoral device, and wirelessly communicate with a controlled device.

The hands-free method is responsible for mapping the user's lower jaw and/or tongue movements and actions to the controlled device movements and actions, either directly or through a pointer or cursor on a display in a manner similar to that of a conventional computer mouse, sensing user's movements and sending the corresponding mapped movements or actions to the controlled device, which will respond accordingly.

The disclosure is suitable, although not limited, to physically handicapped people who do not have full use of their hands. The present disclosure is particularly suitable for use by quadriplegics, amputees and people suffering from severe arthritis or muscular dystrophy. For severe conditions, users have a very high dependency of constant assistance, and the method and apparatus of the present disclosure can significantly reduce the degree of dependency from a helper person, such as a care-taker or a family member.

The method and apparatus are also applied to regular users with no physical limitations as a convenient hands-free and almost-invisible interface. Therefore, it refers to a strong opportunity and trend: the use of “invisibles” (invisible devices) beyond the “wearables”, also related to “Body Area Networks” (BAN).

In a specific embodiment, the disclosure applies to Immersive Reality technologies, more specifically AR/VR HMD's (Head-mounted display) devices. When using such devices user cannot easily reach a touchscreen and has fewer input methods available; for these cases hands-free methods are very convenient for the user to interact with the virtual content while, at same time, having hands available for executing other activities. According to an embodiment, user can control the virtual objects in AR/VR environments by using jaws and tongue movements.

In this embodiment, the controlled equipment is the HMD device wherein the user's jaw and tongue movements are reflected to movements of virtual objects in the Virtual Environment.

### BRIEF DESCRIPTION OF THE DRAWINGS

The objectives and advantages of the present disclosure will become clearer through the following detailed description of the example and non-limitative figures presented at the end of this document, wherein:

FIG. 1 discloses the flowchart of the method of the present disclosure;

FIG. 2 discloses the user's lower jaw movements detection using the intra-oral device;

FIG. 3 discloses the user's tongue movements detection using the intra-oral device;

FIG. 4 illustrates an example of Movement Map UI—User movements versus Controlled device movements settings;

FIG. 5 illustrates an example of mapping user movements and actions to a pointer on display;

FIG. 6 discloses AR/VR embodiment—Mapping user movements and actions to control a virtual object and a pointer on a virtual object; and

FIG. 7 discloses mechanical sensors used for jaws movement detection.

### DETAILED DESCRIPTION

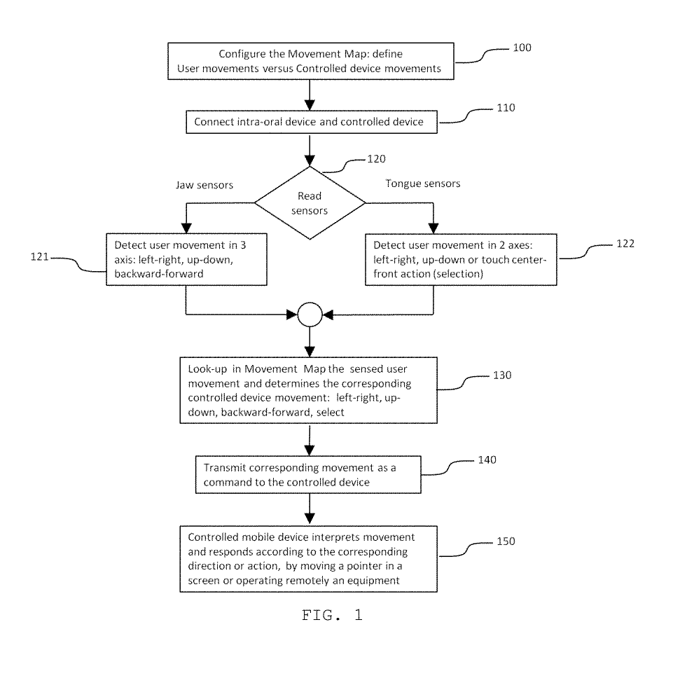

In FIG. 1, it is disclosed the hands-free input method for controlling electronic devices based on user's jaw and tongue movements comprising the following steps:

a) user uses a movement map configuration UI to map lower jaw and tongue movements to the corresponding movements of a controlled device (100);

b) connecting (110) an intra-oral device to the controlled device using a wireless communication method available (200, 300);

c) detecting (120) the jaws' movement by means of jaw sensors (121) and the tongue movement by means of tongue sensors (122) installed in the intra-oral device;

d) the micro-controlled system look-ups the sensed user's jaw movement in the configuration UI (130);

e) transmitting the corresponding movement to the controlled device (140); and

f) the controlled device performs the movement in the corresponding direction or perform the corresponding action (150).

### Sensor Types and Sensing Methods:

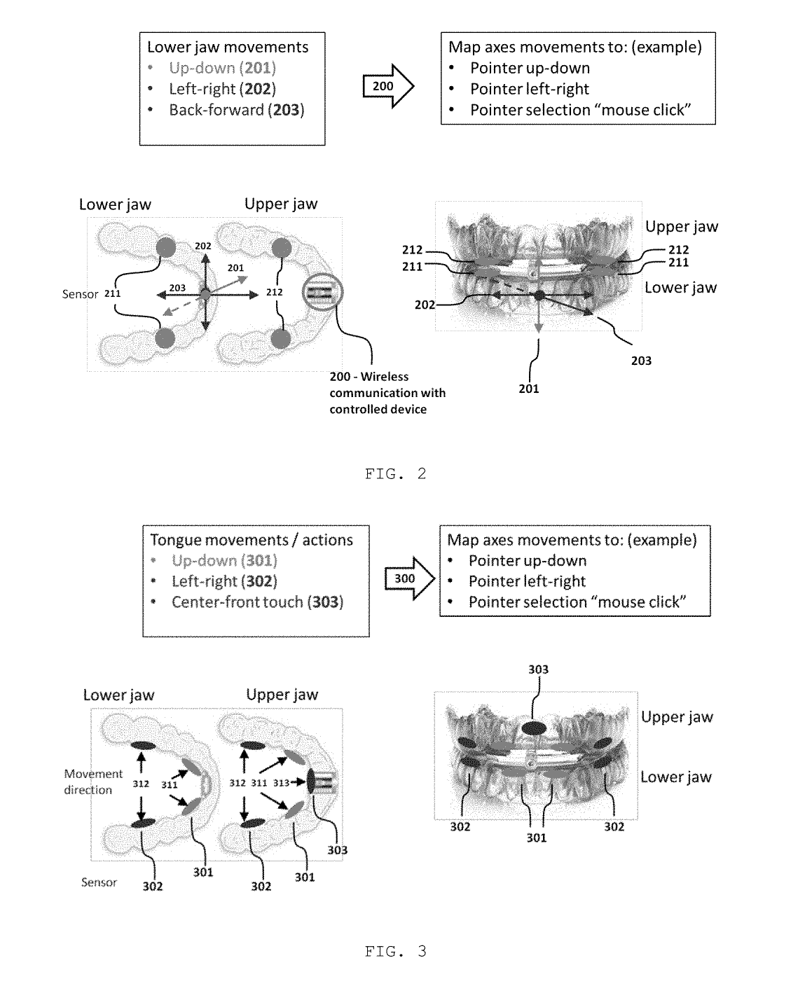

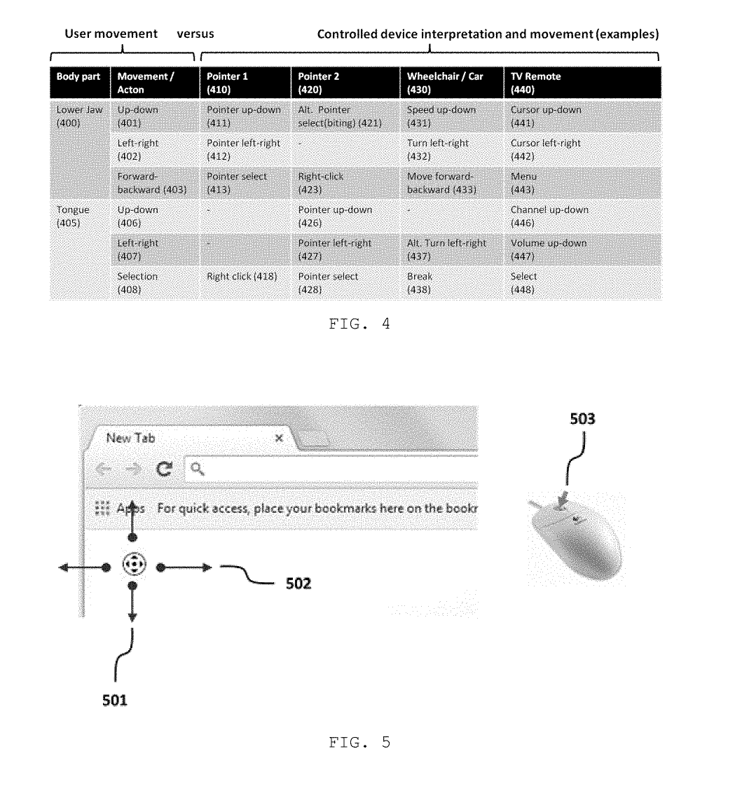

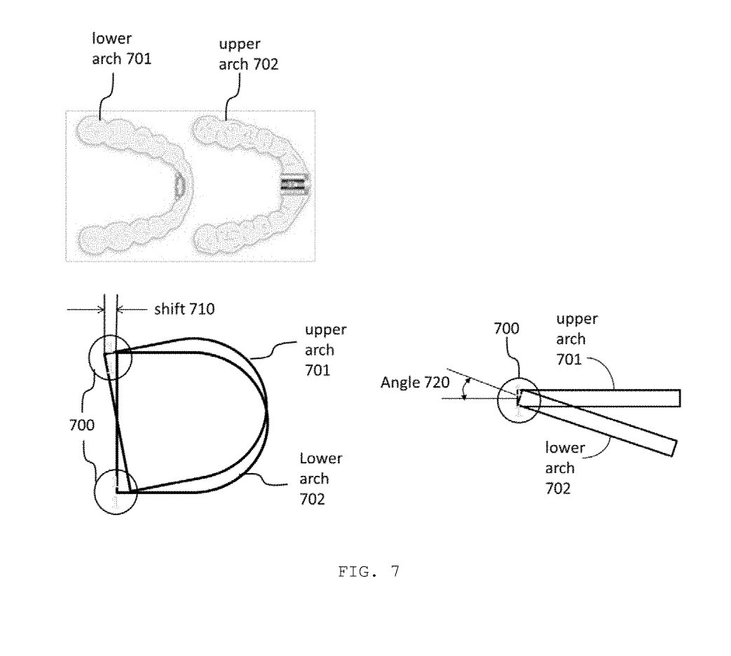

The intra-oral apparatus of the present disclosure can detect both jaws and tongue movements by using different types of sensors: mechanical, electro-magnetic and optical. As illustrated in FIGS. 2 and 3, the following sensing methods can be used to detect, sense and measure respectively the lower jaw and tongue movements:
Lower jaw movements: sensors installed on the top surface of the lower jaws (211) on the bottom surface of the upper jaw (212), and the relative distance and displacement on the 3 axes—left-right (202), up-down (201), back-forward (203) are measured by using electro-magnetic, mechanical or optical methods. Examples of implementations:
optical detection of movement or position of a light source relative to the upper or lower plaque surface, for movements on each of the 3 axes: left-right, up-down, back-forward. This is similar principle used by an optical mouse: using a light source, typically an LED—applied in one of the plaques—and a light detector, such as an array of photodiodes, to detect movement relative to a surface—the other plaque. That allows the detection of slight and short movements, without strain. The concept is analogous to an optical mouse circuitry mounted in one arch, for example the upper arch, of the intraoral device and LED light source will reflect in the other arch surface, for example the lower one, being captured in a light detector (array of photodiodes) mounted in the upper arch. The user jaws movements lift-right and forward-backwards commands the “optical mouse” as usually by user hand on a flat surface. Those movements can analogously drive a pointer up-down or left-right on the screen, or also drive equipment movements, such as a wheelchair, depending on the user mapping settings.
mechanical connections linking the two arches (701, 702) on two joint points at the left and right sides as hinge (700), as disclosed in FIG. 7. Two mechanical sensors can be installed on the joint points of the arches (701, 702), so that the angle (720) of jaws opening up-down can be detected, as well as other relative movements between jaws: left-right, or even back-forwards detected by measuring slight shifts (710) on the joints.
micro-switches push-buttons positioned between the surface of two arches can detect pressure on different positions. A miniaturized version of a micro-switch is installed between jaws surface or in the front of the arch for commanding the selection action, usually performed by a mouse click for example, by the user closing the jaws in order to press the switch. Alternatively, to micro-switches, same principle used by capacitive touchscreens might be applied as an optional method to command the selection action.
Tongue movements: sensors (301, 302, 303) positioned on the internal parts of upper and lower device arches are sensitive to tongue contact, by using electro-magnetic or mechanical methods. See FIG. 3. Examples of implementations:
Capacitance sensors will detect tongue contact with the touch of the tongue, similarly touchscreens senses user finger. Concept is, by using tongue, emulating user finger action when touching on different areas of a capacitive touchscreen. Tongue touches on each of sensors positioned in specific areas of arches (left and right sides of upper arch, left and right sides of lower arch which, in their turn, can be mapped and analogously drive a pointer up-down or left-right on the screen, or also drive equipment movements, such as a wheelchair, depending on the user mapping settings.
Mechanical micro-switches will detect the pressure of the tongue on specific areas of the arches—left-right of both arches (302), front part of upper arch, front part of bottom arch (301). Besides that, a differentiated micro-switch push-button positioned on the up-front area can be used to identify the “select” action (303), equivalent to the mouse click.

### Movements Mapping Configuration—Examples:

In FIG. 4, it is illustrated an example of pointer movements on the display, mostly based on lower jaw movements-action (410), combined with some tongue movements-actions:
pointer up-down=move lower jaw up or down (411);
pointer left-right=move lower jaw to the left or right side (412);
pointer selection (equivalent to the mouse click)=move lower jaw forward-backward (413);
pointer right click (equivalent to the mouse right-click)=touch tongue on the front of the device arches (418).

Following is another example of pointer configuration, mostly based on tongue movements-actions (420), combined with some lower jaw movements-actions:
pointer up-down=touch tongue on the front of the upper device arches, or on the front of the lower device arches (426);
pointer left-right=move tongue to left or right side (427);
pointer selection (equivalent to the mouse click)=touch tongue on the up-front of the device arches (428);
alternative pointer selection (equivalent to the mouse click)=move lower jaw up (421—movement of biting);
pointer right click (equivalent to the mouse right-click)=move lower jaw forward-backward (423).

Alternatively, lower jaw and tongue movement can be mapped to the corresponding behavior of controlled devices which do not necessarily have a display or use a pointer. For example:
Powered wheelchair or car (430): to move forward or backward (433), turn left or right (432, 437), speed up-down (431), break (438);
TV remote control (440): to increase or reduce volume (447), increase or reduce channel number (446), confirm a selected channel o volume level (448), move cursor up-down (441), move cursor left-right (442), click on menu (443);
AR/VR application, to move virtual objects in the immersive environment, without using hands, to left-right, up-down, backwards-forwards, select objects.

### The Device and Method:

The new intra-oral device is composed of an intra-oral device composed of thin plaques covering user's tooth surfaces and attached to the user's upper and lower dental arches, such as a “denture” or braces, which includes sensors installed on the device arches for detecting user's lower jaw and tongue movements and a wireless system for communicating (200, 300) with the controlled device, for example via Bluetooth, Wi-Fi, NFC, mobile networks or any other wireless communication method available, where the device is able to detect the following movements and actions:
lower jaw movements (400), respect to the upper jaw in 3 axis: left-right, up-down, forward-backward (401, 402, 403 and 201, 202, 203);
tongue movements (405): touch sensors positioned on left-right sides, on upper-lower device arches, or on the up-center of the upper device arch (406, 407, 408).

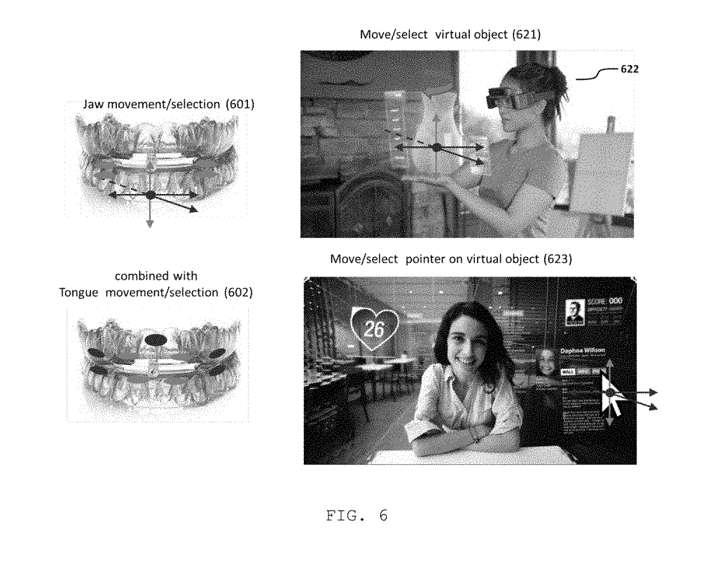

By using a settings configuration UI each of these movements can be mapped to a corresponding pointer movement or action on the display, as disclosed in FIG. 5, for example:
pointer up-down (501);
pointer left-right (502;)
pointer selection—equivalent to the mouse click (503).

Lower jaw and tongue movements can be combined while configuring the pointer movement settings, considering the user preferences, abilities and the most intuitive interpretation when controlling a specific device.

### Physically Handicapped Users:

Disclosure targets the users with severe motion disabilities, which can only move parts of the head, such as tetraplegia or paraplegia which are caused by damages on spinal cord which by birth or due to an accident.

Disclosure can also help users with temporary disabilities, such as users recovering from accidents or post-operatory procedures which impede them to move his arms and hands.

In some degenerative conditions, such as the muscular dystrophy, the person progressively loses movements of most parts of the body. The mouth, responsible for food intake and part of digestive system, is one of the last parts affected, including the movements of the tongue and jaw.

### Non-Handicapped (“Regular”) Users:

Regular users can also take advantage of a new, auxiliary, hands-free interface, in situations or operations where the hands are busy. The proposed intra-oral device is almost invisible, which can be used in cases it is required or desirable a very discrete UI.

The present disclosure provides an opportunity and strong trend: the use of “invisibles” (invisible devices) beyond the “wearables”, also related to “Body Area Networks” (BAN)—BAN devices may be embedded inside the body, implants, may be surface-mounted on the body in a fixed position.

Users Interacting with Virtual Environments (AR/VR):

In a specific embodiment, the disclosure applies to Immersive Reality technologies, as shown in FIG. 6, more specifically to AR/VR HMD's (Head-mounted display) devices (622).

In the preferred embodiment, the controlled equipment is the HMD device wherein the user's jaw and tongue movements and actions (601 and 602) are reflected to movements of virtual objects in the Virtual Environment (621) or a pointer on virtual objects (623).

When using such HMD devices, user cannot easily reach a touchscreen and has fewer input methods available; for these cases hands-free methods are very convenient for the user to interact with the virtual content while, at same time, having hands available for executing other activities. In this embodiment user can control the virtual objects in AR/VR environments by using jaws and tongue movements.

Although the present disclosure has been described in connection with certain preferred embodiments, it should be understood that it is not intended to limit the disclosure to those particular embodiments. Rather, it is intended to cover all alternatives, modifications and equivalents possible within the spirit and scope of the disclosure as defined by the appended claims.

1. A hands-free input method for controlling an electronic device using jaw and tongue movements of a user, the method comprising:
mapping, by a user using a user interface for a configuration of a movement map on the electronic device, jaw and tongue movements of the user to corresponding actions of the electronic device;
connecting, using a wireless connection, an intra-oral device to the electronic device;
detecting jaw and tongue movements of the user using jaw sensors and tongue sensors installed in the connected intra-oral device;
finding the detected jaw and tongue movements of the user in the movement map, and determining, by the electronic device, the corresponding action;
and
performing, by the electronic device, the determined corresponding action.

2. The method according to claim 1, wherein the jaw sensors detect the jaw movements of the user based on a displacement in three dimensions using at least one of an electro-magnetic, mechanical, and optical sensor.

3. The method according to claim 1, wherein the tongue sensors detect the tongue movements based on a displacement in three dimensions using at least one of an electro-magnetic, mechanical, and optical sensor.

4. The method according to claim 1, wherein the action includes at least one of moving a pointer on a display of the electronic device and executing a selection action on the electronic device.

5. The method according to claim 1, wherein the wireless connection includes at least one of Bluetooth, Wi-Fi, Near-Field Communication, and mobile network communication.

6. The method according to claim 2, wherein the jaw sensors include at least one of optical sensors installed on a top surface of a lower jaw of the user, optical sensors installed on a bottom surface of an upper jaw of the user, and mechanical sensors installed on hinges of arches installed in a jaw of the user.

7. The method according to claim 3, wherein the tongue sensors are positioned on the internal parts of upper and lower device arches sensitive to tongue contact.

8. The method according to claim 1, wherein the movement map includes actions taken in a specific context.

9. The method according to claim 1, wherein the electronic device includes at least one of a Virtual Reality or Augmented Reality (AR/VR) device.

10. An apparatus for controlling an electronic device based on jaw and tongue movements of a user, the apparatus comprising:
upper and lower arches attachable to dental arches of the user;
a sensor installed on the upper and lower arches and configured to detect the lower jaw and tongue movements of the user;
a wireless data communicator installed on at least one of the upper and lower arches and configured to connect to the electronic device;
a configuration user interface configured to connect with the electronic device to map the jaw and tongue movements corresponding actions in the electronic device; and
a controller installed in at least one of the upper and lower arches and configured to execute programmable instructions including receiving the detected movements from the sensor, finding the detected movements in the mapped jaw and tongue movements and determining the corresponding action, and wirelessly transmitting the determined action to be executed by the electronic device.
